(19) United States
(12) Patent Application Publication
(10) Pub. No.: US 2003/0027804 A1
(43) Pub. Date: Feb. 6, 2003

(54) Therapeutic combinations for the treatment of hormone deficiencies

(76) Inventor: Roland Gerritsen van der Hoop, Marietta, GA (US)

Correspondence Address: Joseph A. Mahoney, Mayer, Brown & PlaTT, P.O. Box 2828, Chicago, IL, 60690, US

(21) Appl. No.: 09/892,981

(22) Filed: Jun. 27, 2001

(51) Int. Cl. A61K 31/56

(52) U.S. Cl. 514/177; 514/178

(57) ABSTRACT

The present invention relates to methods of treating, preventing, or reducing the risk of developing a male or female menopause disorder or symptom in a mammal by administering to the mammal a sex hormone binding globulin synthesis inhibiting agent and one or more steroids, including, for example, an androgen or an estrogen; to combinations for treating, preventing or reducing the risk of developing a male or female menopause disorder or symptom in a mammal; and to compositions for treating, preventing, or reducing the risk of developing a male or female menopause disorder or symptom in a mammal, where the composition comprises a sex binding globulin synthesis inhibiting agent and one or more steroids, including, for example, an androgen or an estrogen. In addition, the methods, combinations and compositions may be used in conjunction with other pharmaceutical agents aimed at improving sexual performance or impotence, increasing libido, or treating erectile dysfunction, such as VIAGRA®, to enhance their effectiveness.

### FIELD OF THE INVENTION

The present invention relates to methods, combinations, and compositions for treating, preventing, or reducing the risk of developing an androgenic or estrogenic hormone deficiency in a male or female subject, or for treating, preventing, or reducing the risk of developing the symptoms associated with, or related to, an androgenic or estrogenic deficiency in a male or female subject.

### BACKGROUND OF THE INVENTION

Research into “male menopause” or “andropause” shows that there is a drastic drop of serum levels of free testosterone of about 1.5% per year after puberty. While the total testosterone of a male does not drop drastically, the free testosterone, which is the biologically active part of the testosterone, does drop precipitously with aging. In fact, a significant drop of free testosterone can occur as early as the early forties. Studies have shown that men with high testosterone levels live longer, healthier lives and maintain sexual potency. Studies have also shown that testosterone has the ability to stop the spread of breast cancer in females. Additionally, research has shown that testosterone has a protective effect against autoimmune diseases.

The female hormones, estrogen and progesterone, are known to drop drastically to very low levels after menopause. The American College of Physicians and the American College of Obstetricians and Gynecologists has released position papers saying post-menopausal women should seriously consider preventive estrogen/progesterone hormone replacement therapy for their benefit in reducing osteoporosis and heart disease. Maintaining estrogen and progesterone levels has also been shown to improve a number of key risk factors for heart disease in post-menopausal women. The benefits of estrogen/progesterone hormone replenishment therapy include prevention of osteoporosis and heart disease, prevention of vaginal dryness and thinning of the vaginal wall, relief from menopausal symptoms and hot flashes, and the possible benefit of reducing the onset of Alzheimer's disease, dementia, and cataracts. Studies have shown that when estrogen is replenished in conjunction with progesterone, the risks of uterine or breast cancer is nullified.

A. Androgen Synthesis, Metabolism and Regulation

Androgens constitute a group of 19-carbon steroid hormones that exert influence on the male genital tract and are involved with the development and maintenance of secondary male sex characteristics such as beard growth, deepening of the voice at puberty, muscle and bone development, body strength, and sexual drive. Androgens are synthesized in the male testis, in the female ovary, and in the adrenal cortex of both sexes. Once released into the blood, these endogenous androgens serve both as hormones and as prohormones for the formation of two different classes of steroids: 5 alpha-reduced androgens, which act as the intracellular mediators of most actions of the male sex hormones, and estrogens, which enhance some androgenic effects and block others.

Testosterone, the major circulating androgen in men, is synthesized from cholesterol. The approximately 500 million Leydig cells in the testes secrete more than 95% of the 6-7 mg of testosterone produced per day. In women, the ovary and adrenals synthesize small amounts of testosterone. Typically, testosterone is reduced at the 5 alpha position into dihydrotestosterone, which serves as the intracellular mediator of most hormone actions. Although a variety of other naturally occurring androgens have been identified, these are generally weak in potency; and it is now generally believed that they are androgens only to the extent that they can be converted in vivo to testosterone and/or dihydrotestosterone. Two hormones produced by the pituitary gland, luteinizing hormone (“LH”) and follicle stimulating hormone (“FSH”), are required for the development and maintenance of testicular function and negatively regulate testosterone production. Circulating testosterone is metabolized to various 17-keto steroids through two different pathways. Testosterone can be metabolized to dihydrotestosterone (“DHT”) by the enzyme 5α-reductase or to estradiol (“E₂”) by an aromatase enzyme complex.

The gut metabolizes orally administered testosterone and the liver clears about 44% in the first pass. To achieve clinically effective blood levels of testosterone, oral doses as high as 400 mg per day are required. The liver does not as extensively metabolize synthetic androgens, such as methyltestosterone and fluoxymesterone, and thus the synthetic androgens are more suitable for oral administration.

The major metabolites of androgens in urine are physiologically inactive either as free steroids or as water-soluble conjugates. These metabolites are predominantly etiocholanolone, a 5 alpha-reduced metabolite of testosterone; and androsterone, a metabolite of dihydrotestosterone. It is now also recognized that testosterone (but not dihydrotestosterone) can be aromatized into estradiol in a variety of extraglandular tissues, a pathway that accounts for most estrogen synthesis in men and postmenopausal women. The role, if any, of the approximately 50 micrograms of estradiol synthesized each day in normal men has never been defined. Nevertheless, the production of estradiol is considered a normal phenomenon. Experimental evidence suggests that estradiol affects the proliferation of male sex secondary organs, and that estradiol is necessary to induce prostate cancer in animal models.

Testosterone circulates in the blood 98% bound to protein. In men, approximately 40% of the binding is to the high-affinity sex hormone binding globulin (“SHBG”). The remaining 60% is bound weakly to albumin. Thus, a number of measurements for testosterone are available from clinical laboratories. The term “free” testosterone as used herein refers to the fraction of testosterone in the blood that is not bound to protein. The term “total testosterone” or “testosterone” as used herein means the free testosterone plus protein-bound testosterone. The term “bioavailable testosterone” as used herein refers to the non-sex hormone binding globulin bound testosterone and includes that weakly bound to albumin. Plasma concentrations of sex hormone binding globulin determine the ratio of free testosterone to bound testosterone. Total serum testosterone can be measured by assays such as a radioimmunoassay, see, for example, Furuyama et al., Radioimmunoassay for Plasma Testosterone, Steroids, 16:415-428 (1970).

The following table from the UCLA-Harbor Medical Center summarizes the hormone concentrations in normal adult men range:

TABLE 1
Hormone Levels in Normal Men

| Hormone | Normal Range |
|---|---|
| Testosterone | 298 to 1043 ng/dL |
| Free testosterone | 3.5 to 17.9 ng/dL |
| DHT | 31 to 193 ng/dL |
| DHT/T Ratio | 0.052 to 0.33 |
| DHT + T | 372 to 1349 ng/dL |
| SHBG | 10.8 to 46.6 nmol/L |
| FSH | 1.0 to 6.9 mlU/mL |
| LH | 1.0 to 8.1 mlU/mL |
| E₂ | 17.1 to 46.1 pg/mL |

The normal range for total serum testosterone levels in healthy premenopausal women was reported to be 14 to 53.3 ng/dL (Miller et al., Transdermal Testosterone Administration in Women with Acquired Immunodeficiency Syndrome Wasting: A Pilot Study, J. of Clinical Endocrinology and Metabolism, Vol. 83(8): 2717-25 (1998)). Using an ammonium sulfate precipitation method, the normal range of free testosterone levels in normal premenopausal women was reported to be between 1.6 to 12.7 ng/dL (Nankin et al., Daytime Titers of Testosterone, LH, Estrone, Estradiol, and Testosterone-Binding Protein: Acute Effects of LH and LH-Releasing Hormone in Men, J. Clinical Endocrinology Metabolism, Vol. 41:271-81 (1975). Using the equilibrium dialysis method, free testosterone levels measured in healthy premenopausal women was reported to be around 1.3 to 6.8 pg/ml, see, for example, Mather, et al., Free Plasma Testosterone Levels During the Normal Menstrual Cycle. J Endocrinol Invest. October 1985; 8(5):437-41.

There is considerable variation in the half-life of testosterone reported in the literature, ranging from 10 to 100 minutes. Researchers do agree, however, that circulating testosterone has a diurnal variation in normal young men. Maximum levels occur at approximately 6:00 a.m. to 8:00 a.m. with levels declining throughout the day. Characteristic profiles have a maximum testosterone level of 720 ng/dL and a minimum level of 430 ng/dL. The physiological significance of this diurnal cycle, if any, however, is not clear.

B. Testosterone Levels and Sexual Behavior/Performance

Because increasing testosterone concentrations has been shown to alter sexual performance and libido, researchers have investigated methods of delivering testosterone to men. These methods include intramuscular injections (43%), oral replacement (24%), pellet implants (23%), and transdermal patches (10%). A summary of these methods is shown in Table 2.

TABLE 2
Mode of Application and Dosage of Various Testosterone Preparations

| Preparation | Route Of Application | Full Substitution Dose |
|---|---|---|
| In Clinical Use | | |
| Testosterone enanthate | Intramuscular injection | 200-25.0 g |
| | | every 2-3 weeks |
| Testosterone cypionate | Intramuscular injection | 200 mg every 2 weeks |
| Testosterone undecanoate | Oral | 2-4 capsules at 40 mg |
| | | per day |
| Transdermal testosterone | Scrotal skin | 1 membrane per day |
| patch | Non-scrotal skin | 1 or 2 systems per day |
| Transdermal testosterone | Implantation under the | 3-6 implants of |
| patch | abdominal skin | 200 mg every |
| | | 6 months |
| Testosterone implants | | |
| Under Development | | |
| Testosterone cyclodextrin | Sublingual | 2.5-5.0 mg twice daily |
| Testosterone undecanoate | Intramuscular injection | 1000 mg every |
| | | 8-10 weeks |
| Testosterone buciclate | Intramuscular injection | 1000 mg every |
| | | 12-16 weeks |
| Testosterone | Intramuscular injection | 315 mg for 11 weeks |
| microspheres | | |
| Obsolete | | |
| 17α-Methyltestosterone | Oral | 25-5.0 g per day |
| Fluoxymesterone | Sublingual | 10-25 mg per day |
| | Oral | 10-20 mg per day |

C. Hypogonadal Men and Current Treatments for Hypogonadism

Male hypogonadism results from a variety of patho-physiological conditions in which testosterone concentration is diminished below the normal range. The hypogonadic condition is sometimes linked with a number of physiological changes, such as diminished interest in sex, impotence, reduced lean body mass, decreased bone density, lowered mood, and energy levels. Researchers generally classify hypogonadism into one of three types. Primary hypogonadism includes the testicular failure due to congenital or acquired anorchia, XYY Syndrome, XX males, Noonan's Syndrome, gonadal dysgenesis, Leydig cell tumors, maldescended testes, varicocele, Sertoli-Cell-Only Syndrome, cryptorchidism, bilateral torsion, vanishing testis syndrome, orchiectomy, Klinefelter's Syndrome, chemotherapy, toxic damage from alcohol or heavy metals, and general disease (renal failure, liver cirrhosis, diabetes, myotonia dystrophica). Patients with primary hypogonadism show an intact feedback mechanism in that the low serum testosterone concentrations are associated with high FSH and LH concentrations. However, because of testicular or other failures, the high LH concentrations are not effective at stimulating testosterone production.

Secondary hypogonadism involves an idiopathic gonadotropin or LH-releasing hormone deficiency. This type of hypogonadism includes Kallman's Syndrome, Prader-Labhart-Willi's Syndrome, Laurence-Moon-Biedl's Syndrome, pituitary insufficiency/adenomas, Pasqualini's Syndrome, hemochromatosis, hyperprolactinemia, or pituitary-hypothalamic injury from tumors, trauma, radiation, or obesity. Because patients with secondary hypogonadism do not demonstrate an intact feedback pathway, the lower testosterone concentrations are not associated with increased LH or FSH levels. Thus, these men have low testosterone serum levels but have gonadotropins in the normal to low range.

Third, hypogonadism may be age-related. Men experience a slow but continuous decline in average serum testosterone after approximately age 20 to 30 years. Researchers estimate that the decline is about 1-2% per year. Cross-sectional studies in men have found that the mean testosterone value at age 80 years is approximately 75% of that at age 30 years. Because the serum concentration of SHBG increases as men age, the fall in bioavailable and free testosterone is even greater than the fall in total testosterone. Researchers have estimated that approximately 50% of healthy men between the ages of 50 and 70 have levels of bioavailable testosterone that are below the lower normal limit. Moreover, as men age, the circadian rhythm of testosterone concentration is often muted, dampened, or completely lost. The major problem with aging appears to be within the hypothalamic-pituitary unit. For example, researchers have found that with aging, LH levels do not increase despite the low testosterone levels. Regardless of the cause, these untreated testosterone deficiencies in older men may lead to a variety of physiological changes, including sexual dysfunction, decreased libido, loss of muscle mass, decreased bone density, depressed mood, and decreased cognitive function. The net result is geriatric hypogonadism, or what is commonly referred to as “male menopause.”

Today, hypogonadism is the most common hormone deficiency in men, affecting 5 in every 1,000 men. At present, it is estimated that only five percent of the estimated four to five million American men of all ages with hypogonadism currently receive testosterone replacement therapy. Thus, for years, researchers have investigated methods of delivering testosterone to men. These methods include intramuscular injections (43%), oral replacement (24%), pellet implants (23%), and transdermal patches (10%). A summary of these methods is shown in Table 3.

TABLE 3
Mode of Application and Dosage of Various Testosterone Preparations

| Preparation | Route Of Application | Full Substitution Dose |
|---|---|---|
| In Clinical Use | | |
| Testosterone enanthate | Intramuscular injection | 200-25.0 g |
| | | every 2-3 weeks |
| Testosterone cypionate | Intramuscular injection | 200 mg every 2 weeks |
| Testosterone undecanoate | Oral | 2-4 capsules at 40 mg |
| | | per day |
| Transdermal testosterone | Scrotal skin | 1 membrane per day |
| patch | Non-scrotal skin | 1 or 2 systems per day |
| Transdermal testosterone | Implantation under the | 3-6 implants of |
| patch | abdominal skin | 200 mg every |
| | | 6 months |
| Testosterone implants | | |
| Under Development | | |
| Testosterone cyclodextrin | Sublingual | 2.5-5.0 mg twice daily |
| Testosterone undecanoate | Intramuscular injection | 1000 mg every |
| | | 8-10 weeks |
| Testosterone buciclate | Intramuscular injection | 1000 mg every |
| | | 12-16 weeks |
| Testosterone | Intramuscular injection | 315 mg for 11 weeks |
| microspheres | | |
| Obsolete | | |
| 17α-Methyltestosterone | Oral | 25-5.0 g per day |
| Fluoxymesterone | Sublingual | 10-25 mg per day |
| | Oral | 10-20 mg per day |

As discussed below, all of the testosterone replacement methods currently employed suffer from one or more drawbacks, such as undesirable pharmacokinetic profiles or skin irritation. Thus, although the need for an effective testosterone replacement methodology has existed for decades, an alternative replacement therapy that overcomes these problems has never been developed.

Subdermal Pellet Implants

Subdermal implants have been used as a method of testosterone replacement since the 1940s. The implant is produced by melting crystalline testosterone into a cylindrical form. Today, pellet implants are manufactured to contain either 100 mg (length 6 mm, surface area 117 mm²) or 200 mg of testosterone (length 12 mm, surface area 202 mm²). Patients receive dosages ranging from 100 to 1,200 mg, depending on the individual's requirements. The implants are inserted subcutaneously either by using a trocar and cannula or by open surgery into an area where there is relatively little movement. Frequently, the implant is placed in the lower abdominal wall or the buttock. Insertion is made under local anesthesia, and the wound is closed with an adhesive dressing or a fine suture.

Implants have several major drawbacks. First, implants require a surgical procedure that many hypogonadal men simply do not wish to endure. Second, implant therapy includes a risk of extrusion (8.5%), bleeding (2.3%), or infection (0.6%). Scarring is also a risk. Perhaps most important, the pharmacokinetic profile of testosterone pellet implant therapy fails to provide men with a suitable consistent testosterone level. In general, subdermal testosterone implants produce supra-physiologically high serum testosterone levels that slowly decline so that before the next injection subnormally low levels of testosterone are reached. For example, in one recent pharmacokinetic study, hypogonadal patients who received six implants (1,200 mg testosterone) showed an initial short-lived burst release of testosterone within the first two days after application. A stable plateau was then maintained over the next two months (day 2: 1,015 ng/dL; day 63: 990 ng/dL). Thereafter, the testosterone levels declined to baseline by day 300. DHT serum concentrations also rose significantly above the baseline, peaking at about 63 days after implementation and greatly exceeding the upper limit of the normal range. From day 21 to day 189, the DHT/T ratio was significantly increased. The pharmacokinetic profiles for testosterone, DHT, and DHT/T in this study are shown in FIG. 1 of the study. See Jockenhovel et al., Pharmacokinetics and Pharmacodynamics of Subcutaneous Testosterone Implants in Hypogonadal Men, 45 Clinical Endocrinologh 61-71 (1996). Other studies involving implants have reported similar undesirable pharmacokinetic profiles.

Injection of Testosterone Esters

Since the 1950s, researchers have experimented with the intramuscular depot injection of testosterone esters (such as enanthate, cypionate) to increase testosterone serum levels in hypogonadal men. More recent studies have involved injection of testosterone buciclate or testosterone undecanoate in an oil-based vehicle. Other researchers have injected testosterone microcapsule formulations.

Testosterone ester injection treatments suffer from many problems. Patients receiving injection therapy often complain that the delivery mechanism is painful and causes local skin reactions. In addition, testosterone microcapsule treatment requires two simultaneous intramuscular injections of a relatively large volume, which may be difficult to administer due to the high viscosity of the solution and the tendency to block the needle. Other men generally find testosterone injection therapy inconvenient because injection usually requires the patient to visit his physician every two to three weeks.

Equally important, injection-based testosterone replacement treatments still create an undesirable pharmacokinetic profile. The profile generally shows a supra-physiologic testosterone concentration during the first 24 to 48 hours followed by a gradual fall—often to sub-physiologic levels—over then next few weeks. These high serum testosterone levels, paralleled by increases in E₂, are also considered the reason for acne and gynecomastia occurring in some patients, and for polycythaemia, occasionally encountered especially in older patients using injectable testosterone esters. In the case of testosterone buciclate injections, the treatment barely provides normal androgen serum levels and the maximal increase of serum testosterone over baseline does not exceed 172 ng/dL (6 nmol/dL) on average. Because libido, potency, mood, and energy are thought to fluctuate with the serum testosterone level, testosterone injections have largely been unsuccessful in influencing these variables. Thus, testosterone injection remains an undesirable testosterone replacement treatment method.

Oral/Sublingual/Buccal Preparations of Androgens

In the 1970s, researchers began using with oral, sublingual, or buccal preparations of androgens (such as fluoxymesterone, 17 alpha-methyl-testosterone or testosterone undecanoate) as a means for testosterone replacement. More recently, researchers have experimented with the sublingual administration of testosterone-hydroxypropyl-beta-cyclodextrin inclusion complexes. Predictably, both fluoxymesterone and methyl testosterone are 17-alkylated and thus associated with liver toxicity. Because these substances must first pass through the liver, they also produce an unfavorable effect on serum lipid profile, increasing LDL and decreasing HDL, and carbohydrate metabolism. While testosterone undecanoate has preferential absorption through the intestinal lymphatics, it has not been approved in the United States.

The pharmacokinetic profiles for oral, sublingual, and buccal delivery mechanisms are also undesirable because patients are subjected to supra-physiologic testosterone levels followed by a quick return to the baseline. For example, one recent testing of a buccal preparation showed that patients obtained a peak serum hormone levels within 30 minutes after administration, with a mean serum testosterone concentration of 2,688+/−147 ng/dL and a return to baseline in 4 to 6 hours. See Dobs et al., Pharmacokinetic Characteristics, Efficacy and Safety of Buccal Testosterone in Hypogonadal Males: A Pilot Study, 83 J. Clincal Endocrinology & Metabolism 33-39 (1998). To date, the ability of these testosterone delivery mechanisms to alter physiological parameters (such muscle mass, muscle strength, bone resorption, urinary calcium excretion, or bone formation) is inconclusive. Likewise, researchers have postulated that supra-physiologic testosterone levels may not have any extra beneficial impact on mood parameters such anger, nervousness, and irritability.

Testosterone Transdermal Patches

The most recent testosterone delivery systems have involved transdermal patches. Currently, there are three patches used in the market: TESTODERM®, TESTODERM® TTS, and ANDRODERM®.

TESTODERM®

TESTODERM® (Alza Pharmaceuticals, Mountain View, Calif.) was the first testosterone-containing patch developed. The TESTODERM® patch is currently available in two sizes (40 or 60 cm²). The patch contains 10 or 15 mg of testosterone and delivers 4.0 mg or 6.0 mg of testosterone per day. TESTODERM® is placed on shaved scrotal skin, aided by application of heat for a few seconds from a hair dryer.

Studies have also shown that after two to four weeks of continuous daily use, the average plasma concentration of DHT and DHT/T increased four to five times above normal. The high serum DHT levels are presumably caused by the increased metabolism of 5α-reductase in the scrotal skin.

Several problems are associated with the TESTODERM® patch. Not surprisingly, many men simply do not like the unpleasant experience of dry-shaving the scrotal hair for optimal contact. In addition, patients may not be able to wear close-fitting underwear when undergoing treatment. Men frequently experience dislodgment of the patch, usually with exercise or hot weather. In many instances, men experience itching and/or swelling in the scrotal area. Finally, in a number of patients, there is an inability to achieve adequate serum hormone levels.

TESTODERM® TTS

The most recently developed non-scrotal patch is TESTODERM® TTS (Alza Pharmaceuticals, Mountain View, Calif.). It is an occlusive patch applied once daily to the arm, back, or upper buttocks. The system is comprised of a flexible backing of transparent polyester/ethylene-vinyl acetate copolymer film, a drug reservoir of testosterone, and an ethylene-vinyl acetate copolymer membrane coated with a layer of polyisobutylene adhesive formulation. A protective liner of silicone-coated polyester covers the adhesive surface.

Upon application, serum testosterone concentrations rise to a maximum at two to four hours and return toward baseline within two hours after system removal. Many men, however, are unable to obtain and/or sustain testosterone levels within the normal range. The pharmacokinetic parameters for testosterone concentrations are shown below in Table 4:

TABLE 4
TESTODERM ® TTS Testosterone Parameters

| Parameters | Day 1 | Day 5 |
|---|---|---|
| Cₘₐₓ (ng/dL) | 482 ± 149 | 473 ± 148 |
| Tₘₐₓ (h) | 3.9 | 3.0 |
| Cₘᵢₙ (ng/dL) | 164 ± 104 | 189 ± 86 |
| Tₘᵢₙ (h) | 0 | 0 |

Because of TESTODERM® patch is applied to the scrotal skin while the TESTODERM TTS® patch is applied to non-scrotal skin, the two patches provide different steady-state concentrations of the two major testosterone metabolites, DTH and E₂, see Table 5, below:

TABLE 5
Hormone Levels Using TESTODERM ® and TESTODERM ® TTS

| Hormone | Placebo | TESTODERM ® | TESTODERM ® TTS |
|---|---|---|---|
| DHT (ng/dL) | 11 | 134 | 38 |
| E₂ (pg/ml) | 3.8 | 10 | 21.4 |

Likewise, in contrast to the scrotal patch, TESTODERM TTS® treatment creates a DHT/T ratio that is not different from that of a placebo treatment. Both systems, however, suffer from similar problems. In clinical studies, TESTODERM® TTS is associated with transient itching in 12% of patients, erythema in 3% of patients, and puritus in 2% of patients. Moreover, in one 14-day study, 42% of patients reported three or more detachments, 33% of which occurred during exercise.

ANDRODERM®

ANDRODERM® (Watson Laboratories, Inc., Corona, Calif.) is a testosterone-containing patch applied to non-scrotal skin. The circular patch has a total surface area of 37 cm.² The patch consists of a liquid reservoir containing 12.2 mg of testosterone and a permeation-enhanced vehicle containing ethanol, water, monoglycerides, fatty acid esters, and gelling agents. The suggested dose of two patches, applied each night in a rotating manner on the back, abdomen, upper arm, or thigh, delivers 4.1 to 6.8 mg of testosterone.

In general, upon repeated application of the ANDRODERM® patch, serum testosterone levels increase gradually for eight hours after each application and then remain at this plateau level for about another eight hours before declining.

In clinical trials, ANDRODERM® is associated with skin irritation in about a third of the patients, and 10% to 15% of subjects have been reported to discontinue the treatment because of chronic skin irritation. Preapplication of corticosteroid cream at the site of application of ANDRODERM® has been reported to decrease the incidence and severity of the skin irritation. A recent study, however, found that the incidence of skin reactions sufficiently noxious enough to interrupt therapy was as high as 52%. See Parker et al., Experience with Transdermal Testosterone Replacement in Hypogonadal Men, 50 Clinical Endocrinology (oxy) 57-62 (1999). The study reported:

Two-thirds of respondents found the Andropatch unsatisfactory. Patches were variously described as noisy, visually indiscrete, embarrassing, unpleasant to apply and remove, and generally to be socially unacceptable. They fell off in swimming pools and showers, attracted ribald comments from sporting partners, and left bald red marks over trunk and limbs. Dogs, wives, and children were distracted by noise of the patches with body movements. Those with poor mobility or manual dexterity (and several were over 70 years of age) found it difficult to remove packaging an apply patches dorsally.

Transdermal Patch Summary

In sum, the transdermal patch generally offers an improved pharmacokinetic profile compared to other prior art testosterone delivery mechanisms. However, as discussed above, the clinical and survey data shows that all of these patches suffer from significant drawbacks, such as buritus, burn-like blisters, and erythema. Moreover, one recent study has concluded that the adverse effects associated with transdermal patch systems are “substantially higher” than reported in clinical trials. See Parker, supra. Thus, the transdermal patch still remains an inadequate testosterone replacement therapy alternative for most men.

D. Sexual Motivation and Libido

While the terms “sexual performance” and “impotence” describe physiological effects, the terms “sexual motivation” and “libido” describe psychological effects. “Libido” or “sexual motivation” as used herein is a parameter measured by the duration, frequency and extent of sexual daydreams, anticipation of sex, flirting, and sexual interaction.

While doctors now believe that erectile dysfunction is primarily caused by a physiological mechanism, some cases are still attributable to psychological causes. Moreover, decreased libido may also be a reaction to the experience of impotence. Unfortunately, pharmaceuticals such as VIAGRA® treat erectile dysfunction by focusing on the physiological mechanics of attaining and maintaining an erection and do little or nothing to enhance the sexual motivation or libido of men suffering from erectile dysfunction. Thus, there remains a need to treat sexual performance disorders such as impotence in a manner that overcomes both the physiological and psychological problems associated with the disorder.

A number of clinical studies involving testosterone replacement in hypogonadal males have provided convincing evidence that testosterone plays a role in both sexual motivation libido and sexual performance. For example, researchers have reported that testosterone replacement results in increased sexual fantasies, sexual arousal and desire, spontaneous erections during sleep and in the morning, ejaculation, sexual activities with and without a partner, and orgasm through coitus or masturbation. See generally Christiansen, Behavioral Correlates of Testosterone, Testosterone: Action, Deficiency, Substitution 109-111 (1998).

Additionally, testosterone has been shown to lower cholesterol and normalize the abnormal electrocardiograms of patients. Testosterone can also improve diabetic retinopathy as well as lower the insulin requirements of diabetic patients and decrease the percentage of body fat. Administration of testosterone to men has been reported to decrease risk factors for heart attack and low testosterone is also correlated with hypertension, obesity, and increased waist-to-hip ratio.

E. Estradiol Synthesis, Metabolism, and Regulation

Estradiol (1,3,5(10)-Estratriene-3,17 beta-diol) is secreted by the ovary and placenta. It is synthesized by the aromatization of androgens in the thecal and granulosa cells of the ovary and placenta. The aromatization is stimulated by follitropin (FSH). Estradiol synthesis in turn stimulates production of leutinizing hormone (Lutropin or LH) receptors necessary for the synthesis of androgen precursors. Estradiol is important for female sexual differentiation during gestation, sexual development at the onset of puberty, and regulation of the menstrual cycle. The menstrual cycle is the result of a precise coordination of the functional characteristics of the central nervous system, the hypothalamus, the pituitary, the ovary, and the endometrium which regulate the cyclic release of Gonadotropin Releasing Hormone (GnRH), LH and follitropin, and ovarian steroids (estradiol and progesterone). Estradiol is involved in both the stimulation and inhibition of the release of the gonadotropins, exerting both a positive and a negative feedback. Early in the follicular phase, ovarian secretion of estradiol from the thecal and granulosa cells is modest. During the follicular phase, estradiol stimulates endometrial growth (repairing the endometrium after menses). Toward mid-cycle, LH production increases and results in the release of the ovum by the rupture of the developed follicle. After ovulation, estradiol secretion declines slightly. During the luteal phase, estradiol along with progesterone are secreted by the corpus luteum stimulating further endometrial growth. If the ovum is not fertilized, there is a further drop in estradiol and progesterone. This drop in estradiol and progesterone initiates menses. When the ovaries do not function properly due to age, menopause or disease or have been removed, the consequent lack of endogenous estradiol may produce a number of symptoms, such as hot flushes, pain and increased hypocalcemia which may eventually lead to osteoporosis.

Total serum estrogen can be measured by assays known in the art, such as a an ammonium sulfate precipitation assay, see for example, Nankin et al., radioimmunoassay, see, for example, Furuyama et al., (1975). Total serum estrogen refers to the sum of the free estrogen (that is, estrogen unattached to any protein), estrogen weakly bound to serum proteins, such as albumin-bound estrogen, and estrogen tightly bound to high affinity serum proteins, such as sex hormone binding globulin-bound estrogen.

The serum levels of estradiol required for clinical efficacy are in the range of between 40-60 pg/ml. This range of values is the physiologic serum level of the premenopausal women during the early follicular phase.

Estrogenic hormones are currently available in a number of formulations. Transdermal preparations containing estradiol include ERC ALORA®, CLIMARA®, DERMESTRIL®, ESTRADERM®, ESTRADERM® TTS, ESTRADERM MX®, EVOREL®, FEMATRIX®, FEMPATCH®, FEMSEVEN®, MENOREST®, PROGYNOVA® TS, and VIVELLE®. Estrogen gels containing estradiol include ESTROGEL®, and SANDRENA®. Oral preparations of estrogen are also available in a capsule shaped, sugar coated oral tablet, containing 1.25 mg of esterified estrogen and 2.5 mg of methyltestosterone marketed under the brand ESTRATEST® (Solvay Pharmaceuticals, Inc.). A half-strength formulation is also available under the brand ESTRATEST® HS (Solvay Pharmaceuticals, Inc.).

F. Sex Hormone Binding Globulin

The structure and proposed function of sex hormone binding globulin have been described and characterized, see, for example, Rosner et al., Sex Hormone-Binding Globulin Mediates Steroid Hormone Signal Transduction at the Plasma Membrane, J. Steroid Biochem. Mol. Biol. Vol. 69:481-5 (1999). See also, for example, Petra, P. H., The Plasma Sex Steroid Binding Protein (SBP or SHBG): A Critical Review of Recent Developments of the Structure, Molecular Biology, and Function, J. Steroid Biochem. Mol. Biol., Vol. 40:735-53 (1991). A variety of methods have been used to quantify the serum concentrations of sex hormone binding globulin, including ammonium sulfate precipitation, gel filtration, equilibrium dialysis, dextran-coated charcoal, and radioimmunoassay (see, for example, Kahn et al., Radioimmunoassay for Human Testerone-Estradiol Binding Globulin, J. Clinical Endocrinology and Metabolism, Vol. 54:705-710 (1982). The mean serum sex hormone level in healthy premenopausal women is about 84 nmole/L and the normal range is about 36 nmole/L to about 185 nmole/L. Serum sex hormone binding globulin levels are known to be elevated in women treated with oral estrogens, estrogen-containing oral contraceptives, clomiphene, tamoxifen, raloxifene, phenytoin, and sodium valproate, as well as in women who are pregnant, hyperthyroid, have chronic liver disease and HIV infection. See, for example, Bond et al., Sex Hormone Binding Globulin in Clinical Perspective, Acta. Obstet. Gynecol. Scand., Vol. 66:255-262 (1987). See also, for example, Miller et al., (1998).

G. Hormone Replacement Therapy

Various methods or dosage forms are in use or have been proposed for the testosterone and estradiol replacement therapy, for example, tablets, injectable, implants and transdermal devices (patches or gel), etc. Oral therapy, using tablets is well accepted by the patient. However, testosterone and estradiol is rapidly metabolized during the liver first pass. Thus, a high dose of testosterone or estradiol is necessary to achieve clinical appropriate serum levels. Absorption via the gastrointestinal tract results in enhanced delivery of the circulating testosterone or estrogen to the liver, where much of it is metabolized to inactive conjugates, and only a fraction of the active hormone enter general circulation. The liver responds to this enhanced delivery with increased protein and lipid metabolism, and these activities may carry potential risks. Examples of these changes include enhanced hepatic synthesis of renin substrates, sex hormone binding globulin, and changes in cholesterol and lipid lipoprotein metabolism.

The use of parenteral injections and implants or pellets, while they avoid first pass metabolization, are much less convenient for the patient, and are therefore not popular. Testosterone or estradiol transdermal administration is cutaneous delivery that delivers testosterone or estradiol into the systemic circulation via the stratum corneum at a constant rate.

Transdermal administration of drugs offers several therapeutic and compliance advantages over the more traditional routes of administration. A major drawback of this therapy however, is the limitation of the amount of drug that can be transported across the skin. This limitation is due to several factors. Since the skin is a protective barrier by nature, the rates of transport of most compounds through the skin are quite slow. It is generally accepted that a surface of patch beyond 50-100 cm² would result in difficulty of application. Therefore, the application of a transdermal semisolid dosage form such as a gel, cream, ointment, liquid, etc., augment the patient's compliance and the surface of application can be extended.

One effect of estrogen supplementation is an increase in sex hormone binding globulin. Sex hormone binding globulin is a serum protein that binds both testosterone and 17 beta-estradiol, and this binding affects the biological availability of those hormones. Therefore, the increase in sex hormone binding globulin that occurs with estrogen and progestin supplementation results in lower levels of free androgens and estrogens, both of which bind to sex hormone binding globulin, and higher levels of estrogens must be administered in order to achieve the desired biological activity.

All of the testosterone and estradiol replacement methods currently employed, however, suffer from one or more drawbacks. For example, subdermal pellet implants and ester injections are painful and require doctor visits. Many of these methods, such as oral/sublingual/buccal preparations, suffer from undesirable pharmacokinetic profile—creating supra-physiologic testosterone concentrations followed a return to baseline. Transdermal patches provide less than optimal pharmacokinetic characteristics, are embarrassing for many patients, and are associated with significant skin irritation. Thus, although the need for an effective hormone replacement methodology has existed for decades, an alternative replacement therapy that overcomes these problems has never been developed.

### DETAILED DESCRIPTION OF THE INVENTION

While the present invention may be embodied in many different forms, several specific embodiments are discussed herein with the understanding that the present disclosure is to be considered only as an exemplification of the principles of the invention, and it is not intended to limit the invention to the embodiments illustrated.

Where the invention is illustrated herein with particular reference to methyltestosterone, it will be understood that any other inhibitor of the synthesis of sex hormone binding globulin can, if desired, be substituted in whole or in part for methyltestosterone in the methods, combinations and compositions herein described. Where the invention is illustrated herein with particular reference to testosterone, it will be understood that any other steroid in the testosterone anabolic or catabolic pathway can, if desired, be substituted in whole or in part for testosterone in the methods, combinations and compositions herein described. Where the invention is illustrated herein with particular reference to estradiol, it will be understood that any other estrogenic hormone can, if desired, be substituted in whole or in part for estradiol in the methods, combinations and compositions herein described.

The present invention is directed to methods, combinations, and compositions for treating, preventing or reducing the risk of developing an androgenic or estrogenic hormone deficiency, or a male or female menopause disorder, or the symptoms associated with, or related to an androgenic or estrogenic hormone deficiency, or a male or female menopause disorder, in a male or female mammal in need thereof. The method comprises administering to the mammal in a combination therapy an amount of a sex hormone binding globulin synthesis inhibiting agent and one or more steroids, including for example androgen and/or estrogen, where the sex hormone binding globulin synthesis inhibiting agent and the steroid together make a menopause disorder effective amount. The present invention includes methods of halting or slowing the progression of a menopausal disorder once it becomes clinically evident, or treating the symptoms related to the menopausal disorder. The patient may already have a menopausal disorder at the time of administration, or be at risk for developing a menopausal disorder. Also included in the methods, combinations and compositions of the present invention are pharmaceutical compositions comprising a sex hormone binding globulin synthesis inhibiting agent and one or more steroids, including for example androgen and/or estrogen, where the individual agents together make a menopause disorder effective amount. The present invention also includes kits comprising a sex hormone binding globulin synthesis inhibiting agent and one or more steroids, including for example androgen and/or estrogen. The kits also contain a set of instructions for the patient. When administered as part of a combination therapy, the sex hormone binding globulin synthesis inhibiting agent together with the steroid provide enhanced treatment options for treating menopause in a mammal as compared to administration of either a sex hormone binding globulin synthesis inhibiting agent or a steroid alone.

Besides being useful for human treatment, the present invention is also useful for veterinary treatment of companion mammals, exotic animals and farm animals, including mammals, rodents, and the like. In one embodiment, the mammals include horses, dogs, and cats.

Contemplated methods, combinations and compositions are useful to treat a variety of menopausal disorders, or the symptoms associated with, or related to a menopausal disorder, including but not limited to hypogonadism, sexual or erectile dysfunction, decreased libido, hyperglycemia, hyperglyceridemia, hypercholesterolemia, hypertension, atherosclerosis, cardiovascular disorders and diseases, vasomotor symptoms, obesity, diabetes, osteoporosis, osteopenia, vaginal dryness, thinning of the vaginal wall, relieving menopausal symptoms and hot flashes, improving cognitive dysfunction, Alzheimer's disease, dementia, cataracts, cervical cancer, uterine cancer, breast cancer, Klinefelter's syndrome, teratogenic disorder, and cervical dysplasia.

The phrase “combination therapy” embraces the administration of a sex hormone binding globulin synthesis inhibiting agent and one or more steroids, including for example, androgen and/or estrogen, as part of a specific treatment regimen intended to provide a beneficial effect from the co-action of these therapeutic agents for the treatment of menopause. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days, weeks, months or years depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, where each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule or tablet having a fixed ratio of each therapeutic agent or in multiple, single capsules, tablets, or gels for each of the therapeutic agents. Sequential or substantially simultaneous administration of each therapeutic agent can be effected by any appropriate route including, but not limited to, oral routes, percutaneous routes, intravenous routes, intramuscular routes, and direct absorption through mucous membrane tissues. The therapeutic agents can be administered by the same route or by different routes. For example, a first therapeutic agent of the combination selected may be administered orally, while the other therapeutic agents of the combination may be administered percutaneously. Alternatively, for example, all therapeutic agents may be administered orally, or all therapeutic agents may be administered percutaneously, or all therapeutic agents may be administered intravenously, or all therapeutic agents may be administered intramuscularly, or all therapeutic agents can be administered by direct absorption through mucous membrane tissues. The sequence in which the therapeutic agents are administered is not narrowly critical. “Combination therapy” also can embrace the administration of the therapeutic agents as described above in further combination with other biologically active ingredients, such as, but not limited to, agents for improving sexual performance or impotence, including agents to treat erectile dysfunction, such as VIAGRA®, or increasing libido by increasing testosterone levels in men, and non-drug therapies, such as, but not limited to, surgery.

The phrase “menopause disorder” or “menopausal disorder” encompasses both male “andropausal” and female menopausal disorders, and includes either a peri-menopausal condition or a post-menopausal condition. “Peri-menopausal condition” refers to a condition that occurs either during menopausal onset, or prior thereto at a time when menopausal onset normally occurs, and either is caused by menopausal onset or has a greater than random coincidence therewith. Peri-menopausal conditions include, for example, hot flashes and reduction of bone mass. “Post-menopausal condition” refers to a condition that occurs after menopausal onset, and either is caused by menopause or has a greater than random coincidence therewith. Post-menopausal conditions include, for example, vasomotor symptoms, osteopenia, osteoporosis, cardiovascular disease and cognitive dysfunction.

A “menopause disorder effect” or “menopause disorder effective amount” is intended to qualify the amount of a sex hormone binding globulin synthesis inhibiting agent and a steroid, including for example androgen and/or estrogen, required to treat or prevent a menopause disorder or relieve to some extent one or more of the symptoms of a menopause disorder, including, but not limited to, normalizing hypogonadism, improving sexual or erectile dysfunction, increasing libido, lowering blood cholesterol levels; normalizing abnormal electrocardiograms of patients and improving vasomotor symptoms; improving diabetic retinopathy as well as lowering the insulin requirements of diabetic patients; decreasing the percentage of body fat; decreasing the risk factors for heart attack, hypertension, and obesity; preventing osteoporosis, osteopenia, vaginal dryness, and thinning of the vaginal wall; relieving menopausal symptoms and hot flashes; improving cognitive dysfunction; treating or reducing the onset of cardiovascular disease, Alzheimer's disease, dementia, and cataracts; and treating or reducing the risk of cervical, uterine or breast cancer.

The phrase “non oral” or “non orally deliverable” refers to percutaneous, transmucosal, implantation, inhalation spray, rectal, vaginal, topical, buccal (for example, sublingual), or parenteral (for example, subcutaneous, intramuscular, intravenous, intramedullary and intradermal injections, or infusion techniques) formulations and administrations.

The use of the term “about” in the present disclosure means “approximately,” and use of the term “about” indicates that dosages slightly outside the cited ranges may also be effective and safe, and such dosages are also encompassed by the scope of the present claims.

The phrase “pharmaceutically acceptable” is used adjectivally herein to mean that the modified noun is appropriate for use in a pharmaceutical product. Pharmaceutically acceptable cautions include metallic ions and organic ions. More preferred metallic ions include, but are not limited to appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

The phrase “penetration enhancer” refers to an agent known to accelerate the delivery of the drug through the skin. These agents also have been referred to as accelerants, adjuvants, and sorption promoters, and are collectively referred to herein as “enhancers.” This class of agents includes those with diverse mechanisms of action including those which have the function of improving the solubility and diffusibility of the drug, and those which improve percutaneous absorption by changing the ability of the stratum corneum to retain moisture, softening the skin, improving the skin's permeability, acting as penetration assistants or hair-follicle openers or changing the state of the skin such as the boundary layer. The penetration enhancer of the present invention is a functional derivative of a fatty acid, which includes isosteric modifications of fatty acids or non-acidic derivatives of the carboxylic functional group of a fatty acid or isosteric modifications thereof. In one embodiment, the functional derivative of a fatty acid is an unsaturated alkanoic acid in which the —COOH group is substituted with a functional derivative thereof, such as alcohols, polyols, amides and substituted derivatives thereof. The term “fatty acid” means a fatty acid that has four (4) to twenty-four (24) carbon atoms.

Non-limiting examples of penetration enhancers include C₈-C₂₂ fatty acids such as isostearic acid, octanoic acid, and oleic acid; C₈-C₂₂ fatty alcohols such as oleyl alcohol and lauryl alcohol; lower alkyl esters of C₈-C₂₂ fatty acids such as ethyl oleate, isopropyl myristate, butyl stearate, and methyl laurate; di(lower)alkyl esters of C₆-C₂₂ diacids such as diisopropyl adipate; monoglycerides of C₈-C₂₂ fatty acids such as glyceryl monolaurate; tetrahydrofurfuryl alcohol polyethylene glycol ether; polyethylene glycol, propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes.

The thickeners used herein may include anionic polymers such as polyacrylic acid (CARBOPOL® by B.F. Goodrich Specialty Polymers and Chemicals Division of Cleveland, Ohio), carboxymethylcellulose and the like. Additional thickeners, enhancers and adjuvants may generally be found in Remington's The Science and Practice of Pharmacy, Meade Publishing Co., United States Pharmacopeia/National Formulary.

As used herein, the term “lower alcohol,” along or in combination, means a straight-chain or branched-chain alcohol moiety containing one to about six carbon atoms. In one embodiment, the lower alcohol contains one to about 4 carbon atoms, and in another embodiment the lower alcohol contains two to about 3 carbon atoms. Examples of such alcohol moieties include methanol, ethanol, n-propanol, isopropanol, n-butanol, isobutanol, sec-butanol, and tert-butanol.

As used herein, the term “lower alkyl”, alone or in combination, means a straight-chain or branched-chain alkyl radical containing one to about six carbon atoms. In one embodiment, the lower alkyl contains one to about four carbon atoms. Examples of such radicals include methyl, ethyl, n-propyl, isopropyl, n-butyl, isobutyl, sec-butyl, and tert-butyl.

The compositions of the present invention are used in a “pharmacologically effective amount.” This means that the concentration of the therapeutic agents of the present invention are such that in the composition it results in a therapeutic level of drug delivered over the term that the drugs are to be used. Such delivery is dependent on a number of variables including the time period for which the individual dosage unit is to be used, the flux rate of the therapeutic agent, for example, testosterone or estradiol, from the gel, surface area of application site, etc. The amount of therapeutic agent, for example, testosterone or estradiol, necessary can be experimentally determined based on the flux rate of the drug through the gel, and through the skin when used with and without enhancers. It is understood, however, that specific dose levels of the therapeutic agents of the present invention for any particular patient depends upon a variety of factors including the activity of the specific compound employed, the age, body weight, general health, sex, and diet of the patient, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of menopause in accordance with the present invention. In terms of treatment protocols, it should be appreciated that the dosage to be administered will depend on several factors, including the particular agent that is administered, the route administered the condition of the particular patient, etc. Generally speaking, one will desire to administer an amount of the compound that is effective to achieve a serum level commensurate with the concentrations found to be effective in vitro. Thus, where an compound is found to demonstrate in vitro activity at, for example, 10 ng/ml, one will desire to administer an amount of the drug that is effective to provide about a 10 ng/ml concentration in vivo. Determination of these parameters is well within the skill of the art. These considerations, as well as effective formulations and administration procedures are well known in the art and are described in standard textbooks.

Illustratively, for an adult human, a therapeutically effective amount of methyltestosterone per daily dose for use in the present invention is typically about 0.2 mg to about 5.0 mg; a therapeutically effective amount of testosterone per daily unit for use in the present invention is typically about 0.1 mg to about 10 mg; and a therapeutically effective amount of estradiol per daily dose for use in the present invention is typically about 0.1 mg to about 10 mg.

The present invention includes compounds that inhibit the synthesis of the sex hormone binding globulin. Sex hormone binding globulin is a serum protein, and is known to bind to testosterone and estradiol, effecting the biological activity of these hormones. Specific compounds of interest that inhibit the synthesis the sex hormone binding globulin include but are not limited to methyltestosterone and fluoxymesterone, and all salts, esters, amides, enantiomers, isomers, tautomers, prodrugs and derivatives of these compounds. Methyltestosterone is currently available in various formulations including those available orally, for example ANDROID® and TESTRED® ICN. Fluoxymesterone is also currently available in various formulations including those available orally, for example HALOSTESTIN®. Combinations of the above mentioned compounds can be used.

While not wishing to be bound by theory, it is believed that methyltestosterone decreases hepatic synthesis of endogenous proteins like sex hormone binding globulin. This decrease in synthesis produces a decline in blood concentrations of sex hormone binding globulin, which is the primary means of endogenous hormone transport. The decrease in sex hormone binding globulin subsequently causes an increase in free-horrnone concentration for binding at the receptor. Transdermal application of an androgen, for example, testosterone, or an estrogen, for example, estradiol, bypasses first-pass metabolism and can provide a means of increasing hormone concentrations in the bloodstream. Thus, in combination therapy, methyltestosterone and percutaneously administered testosterone and/or estradiol produce a greater therapeutic effect and provide a means of increasing hormone concentrations in the bloodstream. Methyltestosterone and testosterone and/or estradiol produce a greater therapeutic effect than either entity alone because the decrease in hormone binding ability is coupled with an increased hormone bioavailability, producing higher free-hormone concentrations that would be produced by methyltestosterone alone, or testosterone alone, or estradiol alone.

Certain formulations of the invention will contain from about 0.2 mg to about 50.0 mg methyltestosterone or the equivalent per dosage unit. The formulations may contain for example, about 0.2, 0.3, 0.4, 0.5, 0.6, 0.7, 0.8, 0.9, 1.0, 1.1, 1.2, 1.3, 1.4, 1.5, 2.0, 2.5, 3.0, 3.5, 4.0, 4.5, 5.0, 10.0, 20.0, 30.0, 40.0 or 50.0 mg methyltestosterone per dosage unit.

A class of androgens useful in the methods, combinations and compositions of the present invention include steroids in the testosterone anabolic or catabolic pathway. In a broad aspect of the invention, the active ingredients employed in the composition may include anabolic steroids such as androisoxazole, bolasterone, clostebol, ethylestrenol, formyldienolone, 4-hydroxy-19-nortestosterone, methenolone, methyltrienolone, nandrolone, oxymesterone, quinbolone, stenbolone, trenbolone; androgenic steroids such as boldenone, fluoxymesterone, mestanolone, mesterolone, methandrostenolone, 17-methyltestosterone, 17 alpha-methyl-testosterone 3-cyclopentyl enol ether, norethandrolone, normethandrone, oxandrolone, oxymetholone, prasterone, stanlolone, stanozolol, dihydrotestosterone, testosterone; and progestogens such as anagestone, chlormadinone acetate, delmadinone acetate, demegestone, dimethisterone, dihydrogesterone, ethinylestrenol, ethisterone, ethynodiol, ethynodiol diacetate, flurogestone acetate, gestodene, gestonorone caproate, haloprogesterone, 17-hydroxy-16-methylene-progesterone, 17 alpha-hydroxyprogesterone, 17 alpha-hydroxyprogesterone caproate, medrogestone, medroxyprogesterone, megestrol acetate, melengestrol, norethindrone, norethindrone acetate, norethynodrel, norgesterone, norgestimate, norgestrel, norgestrienone, 19-norprogesterone, norvinisterone, pentagestrone, progesterone, promegestone, quingestrone, and trengestone; and all salts, esters, amides, enantiomers, isomers, tautomers, prodrugs and derivatives of these compounds. (Based upon the list provided in The Merck Index, Merck & Co. Rahway, N.J. (1998)).

Testosterone is currently available in various formulations including, but not limited to those available as an injectable, for example DEPO-TESTOSTERONE® (testosterone cypionate), and DELATESTRYL BTG® (testosterone enanthate), as a transdermal, for example TESTODERM® (testosterone), TESTODERM® TTS (testosterone), and ANDRODERM®, or as a gel. One such testosterone gel that may be used in the methods, combinations and compositions of the present invention has only recently been made available in the United States under the trademark ANDROGEL® by Unimed Pharmaceuticals, Inc., Deerfield, Ill., a wholly owned subsidiary of Solvay Pharmaceuticals, Inc.

Combinations of the above mentioned androgens can be used.

In one embodiment, the testosterone gel is comprised of the following substances in approximate amounts:

TABLE 6
Composition of ANDROGEL ®

| | AMOUNT (w/w) |
|---|---|
| SUBSTANCE | PER 100 g OF GEL |
| Testosterone | 1.0 g |
| Carbopol 980 | 0.90 g |
| Isopropyl myristate | 0.50 g |
| 0.1 N NaOH | 4.72 g |
| Ethanol (95% w/w) | 72.5 g* |
| Purified water (qsf) | 100 g |

*Corresponding to 67 g of ethanol.

One skilled in the art will appreciate that the constituents of this formulation may be varied in amounts yet continue to be within the spirit and scope of the present invention. For example, the composition may contain about 0.1 to about 10.0 g of testosterone, about 0.1 to about 5.0 g CARBOPOL, about 0.1 to about 5.0 g isopropyl myristate, and about 30.0 to about 98.0 g ethanol.

A therapeutically effective amount of the gel is rubbed onto a given area of skin by the user. The combination of the lipophilic testosterone with the hydroalcoholic gel helps drive the testosterone in to the outer layers of the skin where it is absorbed and then slowly released into the blood stream. As demonstrated by the data presented herein, the administration of the gel of the present invention has a sustained effect.

In another embodiment, the androgen is testosterone and is formulated for percutaneous administration comprising testosterone in a hydroalcoholic gel. The gel comprises one or more lower alcohols, a penetration enhancing agent, a thickener, and water. Additionally, the present invention may optionally include salts, emollients, stabilizers, antimicrobials, fragrances, and propellants.

Certain formulations of the invention will contain from about 0.1 mg to about 100 mg testosterone or the equivalent per dosage unit. The formulations may contain for example, about 0.1, 0.25, 0.5, 0.625, 1.0, 1.25, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 20.0, 30.0, 40.0, 50.0 or 100.0 mg testosterone per dosage unit.

Another class of agents useful in the methods, combinations and compositions of the present invention include estrogenic hormones. In one embodiment, the estrogenic hormone is the naturally occurring estrogen 17 beta-estradiol (beta-estradiol; 1,3,5(10)-estratriene-3,17 beta-diol). Other estrogenic steroid hormones can be used in partial or complete replacement of 17 beta-estradiol, for example, an ester which is biologically compatible and can be absorbed effectively transdermally. The estradiol esters can be, illustratively estradiol-3,17-diacetate; estradiol-3-acetate; estradiol-17-acetate; estradiol-3,17-divalerate; estradiol-3-valerate; estradiol-17-valerate; 3-mono, 17-mono and 3,17-dipropionate esters, corresponding cypionate, heptanoate, benzoate and the like esters; ethynil estradiol; estrone and other estrogenic steroids and salts, enantiomers, isomers, tautomers, prodrugs and derivatives thereof that are possible to administer by transdermal route. Other estrogen-related compounds that may be used in the methods, combinations and compositions of the present invention include, but are not limited to conjugated estrogens (including estrone sulfate, equilin, and 17-.alpha.-dihydroequilin), estradiol valerate, estriol, estrone, estrone sulfate, estropipate, ethinyl estradiol, mestranol, and all salts, esters, amides, enantiomers, isomers, tautomers, prodrugs and derivatives of these compounds.

Estrogenic hormones are currently available in various formulations including, but not limited to those available as a cream, pessary, vaginal ring, vaginal tablet, transdermal preparation, gel, and oral tablet. Examples of vaginal creams include PREMARIN® (conjugated estrogen), ORTHO DIENOSTEROL® (dienosterol), and OVESTIN® (estriol). Available pessary formulations include ORTHO-GYNEST® (estriol), and TAMPOVAGAN® (stilbestrol). An example of a vaginal ring formulation is ESTRING® (estradiol), and an example of a vaginal tablet is VAGIFEM® (estradiol). Available transdermal estrogen preparations containing estradiol include ERC ALORA®, CLIMARA®, DERMESTRIL®, ESTRADERM®, ESTRADERM® TTS, ESTRADERM® MX, EVOREL®, FEMATRIX®, FEMPATCH®, FEMSEVEN®, MENOREST®, PROGYNOVA® TS, and VIVELLE®. Available estrogen gels containing estradiol include ESTROGEL®, and SANDRENA®. Estradiol is also available formulated as an implant pellet, for example, ESTRADIOL IMPLANT®. Tablet formulations include PREMARIN® (conjugated estrogen), ESTRATAB® (esterified estrogen), ESTRATEST® (esterified estrogen, methyltestosterone), MENEST® (esterified estrogen), CLIMAGEST®, (estradiol), CLIMAVAL® (estradiol), ELLESTE SOLO® (estradiol), ESTRACE® (estradiol), PROGYNOVA® (estradiol), ZUMENON® (estradiol), HORMONIN® (estradiol, estrone, estriol), HARMOEN® (estrone), OGEN® (estropipate), and ORTHO-EST® (estropipate).

Combinations of the above mentioned estrogenic hormones can be used.

In one embodiment, the estrogenic hormone is formulated for percutaneous administration in a hydroalcoholic gel. The gel comprises one or more lower alcohols; a penetration enhancing agent; a thickener; and water. Additionally, the present invention may optionally include salts, emollients, stabilizers, antimicrobials, fragrances, and propellants. In one embodiment, the estrogenic gel is comprised of the following substances in approximate amounts:

TABLE 7
Composition of Estradiol Gel

| | AMOUNT (w/w) |
|---|---|
| SUBSTANCE | PER 100 g OF GEL |
| 17-beta-oestradiol | 0.06 g |
| Carbopol 980 | 1.0 g |
| Triethanolamine | 1.35 g |
| Ethanol (95% w/w) | (59 ml) |
| Purified water (qsf) | 100 g |

One skilled in the art will appreciate that the constituents of this formulation may be varied in amounts yet continue to be within the spirit and scope of the present invention. For example, the composition may contain about 0.1 to about 10.0 g of estradiol, about 0.1 to about 5.0 g CARBOPOL, about 0.1 to about 5.0 g triethanolamine, and about 30.0 to about 98.0 g ethanol.

A therapeutically effective amount of the gel is rubbed onto a given area of skin by the user. The combination of the lipophilic estradiol with the hydroalcoholic gel helps drive the estradiol in to the outer layers of the skin where it is absorbed and then slowly released into the blood stream. It is contemplated that the administration of the gel of the present invention has a sustained effect.

Certain formulations of the invention will contain from about 0.1 mg to about 100 mg estradiol or the equivalent per dosage unit. The formulations may contain for example, about 0.1, 0.25, 0.5, 0.625, 1.0, 1.25, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 20.0, 30.0, 40.0, 50.0 or 100.0 mg estradiol per dosage unit.

For additional examples of other compositions containing estrogen-related compounds or androgen-related compounds that may be used in the methods, combinations and compositions of the present invention, see for example, Sturdee, D. W., et al. Br. J. Obstet. Gynecol. (1997) 104:109-115.

Toxicity and therapeutic efficacy of the therapeutic agents of the present invention can be determined by standard pharmaceutical procedures, for example, for determining LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

As discussed above, combination therapy also can embrace the administration of the therapeutic agents as described above in further combination with other biologically active ingredients, such as, but not limited to, agents for improving sexual performance or impotence, and include agents to treat erectile dysfunction, or increasing libido by increasing testosterone levels in men, such as VIAGRA®. Other pharmaceuticals useful for treating erectile dysfunction include any agent that is effective to inhibit the activity of a phosphodiesterase. Suitable phosphodiesterase inhibitors include, but are not limited to, inhibitors of the type III phosphodiesterase (cAMP-specific-cGMP inhibitable form), the type IV phospodiesterase (high affinity-high specificity cAMP form) and the type V phosphodiesterase (the cGMP specific form). Additional inhibitors that may be used in conjunction with the present invention are cGMP-specific phosphodiesterase inhibitors other than type V inhibitors.

Examples of type III phospodiesterase inhibitors that may be administered include, but are not limited to, bypyridines such as milrinone and amirinone, imidazolones such as piroximone and enoximone, dihydropyridazinones such as imazodan, 5-methyl-imazodan, indolidan and ICI1118233, quinolinone compounds such as cilostamide, cilostazol and vesnarinone, and other molecules such as bemoradan, anergrelide, siguazodan, trequinsin, pimobendan, SKF-94120, SKF-95654, lixazinone and isomazole.

Examples of type IV phosphodiesterase inhibitors suitable herein include, but are not limited to, rolipram and rolipram derivatives such as RO20-1724, nitraquazone and nitraquazone derivatives such as CP-77059 and RS-25344-00, xanthine derivatives such as denbufylline and ICI63197, and other compounds such as EMD54622, LAS-31025 and etazolate.

Examples of type V phosphodiesterase inhibitors include, but are not limited to, zaprinast, MY5445, dipyridamole, and sildenafil. Other type V phosphodiesterase inhibitors are disclosed in PCT Publication Nos. WO 94/28902 and WO 96/16644. In the preferred embodiment, an inhibitor of phosphodiesterase type 5 (“PDE5”), such as VIAGRA® (sildenafil citrate USP) is used.

Other compounds useful for treating erectile dysfunction may also be used. These include: (a) pentoxifylline (TRENTAL®); (b) yohimbine hydrocholoride (ACTIBINE®, YOCON®, YOHIMEX®); (c) apomorphine (UPRIMA®); (d) alprostadil (the MUSE® system, TOPIGLAN®, CAVERJECT®); (e) papavaerine (PAVABID®, CERESPAN®); (f) phentolamine (VASOMAX®, REGITINE®), and combinations, salts, prodrugs, isomers, amides, esters, tautomers, derivatives and enantiomers of all of the above.

The compounds described in PCT Publication No. WO 94/28902 are pyrazolopyrimidinones. Examples of the inhibitor compounds include 5-(2-ethoxy-5-morpholinoacetylphenyl)-1-methyl-3-n-propyl-1,6-dihydro-7H-pyrazolo[4,3-d]pyrimidin-7-one, 5-(5-morpholinoacetyl-2-n-propoxyphenyl)-1-methyl-3-n-propyl-1,6-dihydro-7-H-pyrazolo[4,3-d]pyrimidin-7-one, 5-[2-ethoxy-5-(4-methyl-1-piperazinylsulfonyl)-phenyl]1-methyl-3-n-propyl-1,6-dihydro-7H-pyrazolo[4,3-d]pyrimidin-7-one, 5-[2-allyloxy-5-(4-methyl-1-piperazinylsulfonyl)-phenyl]-1-methyl-3-n-propyl-1,6-dihydro-7H-pyrazolo[4,3-d]pyrimidin-7-one, 5-[2-ethoxy-5-[4-(2-propyl)-1-piperazinylsulfonyl)-phenyl]-1-methyl-3-n-propyl-1,6dihydro-7H-pyrazolo[4,3-d]pyrimidin-7-one, 5-[2-ethoxy-5-[4-(2-hydroxyethyl)-1-piperazinylsulfonyl)phenyl]-1-methyl-3-n-propyl-1,6-dihydro-7H-pyrazolo[4,3-d]pyrimidin-7-one, 5-[5-[4-(2-hydroxyethyl)-1-piperazinylsulfonyl]-2-n-propoxyphenyl]-1-methyl 1-3-n-propyl-1,6-dihydro-7H-pyrazolo[4,3-d]pyrimidin-7-one, 5[2-ethoxy-5-(4-methyl-1-piperazinylcarbonyl)phenyl]-1-methyl-3-n-propyl-1,6-dihydro-7H-pyrazolo[4,3-d]pyrimidin-7-one, and 5-[2-ethoxy-5-(1-methyl-2-imidazolyl)phenyl]-1-methyl-3-n-propyl-1,6-dihydro-7H-pyrazolo[4,3-d]pyrimidin-7-one.

The phosphodiesterase inhibitors described in PCT Publication No. WO 96/16644 include griseolic acid derivatives, 2-phenylpurinone derivatives, phenylpyridone derivatives, fused and condensed pyrimidines, pyrimidopyrimidine derivatives, purine compounds, quinazoline compounds, phenylpyrimidinone derivative, imidazoquinoxalinone derivatives or aza analogues thereof, phenylpyridone derivatives, and others. Specific examples of the phosphodiesterase inhibitors disclosed in WO 96/16644 include 1,3-dimethyl-5-benzylpyrazolo[4,3-d]pyrimidine-7-one, 2-(2-propoxyphenyl)-6-purinone, 6-(2-propoxyphenyl)-1,2-dihydro-2-oxypyridine-3-carboxamide, 2-(2-propoxyphenyl)-pyrido[2,3-d]pyrimid-4(3H)-one, 7-methylthio-4-oxo-2-(2-propoxyphenyl)-3,4-dihydro-pyrimido[4,5-d]pyrimidine, 6-hydroxy-2-(2-propoxyphenyl)pyrimidine-4-carboxamide, 1-ethyl-3-methylimidazo[1,5a]quinoxalin-4(5H)-one, 4-phenylmethylamino-6-chloro-2-(1-imidazoloyl)quinazoline, 5-ethyl-8-[3-(N-cyclohexyl-N-methylcarbamoyl)-propyloxy]-4,5-dihydro-4-oxo-pyrido[3,2-e]-pyrrolo[1,2-a]pyrazine, 5′-methyl-3′-(phenylmethyl)-spiro[cyclopentane-1,7′(8′H)-(3′H)-imidazo[2,1b]purin]4′(5′H)-one, 1-[6-chloro-4-(3,4-methylenedioxybenzyl)-aminoquinazolin-2-yl)piperidine-4-carboxylic acid, (6R, 9S)-2-(4-trifluoromethyl-phenyl)methyl-5-methyl-3,4,5,6a,7,8,9,9a-octahydrocyclopent[4,5]-midazo[2,1-b]-purin-4-one, 1t-butyl-3-phenylmethyl-6-(4-pyridyl)pyrazolo[3,4-d]-pyrimid-4-one, 1-cyclopentyl-3-methyl-6-(4-pyridyl)-4,5-dihydro-1H-pyrazolo[3,4-d]pyrimid-4-one, 2-butyl-1-(2-chlorobenzyl)6-ethoxy-carbonylbenzimidaole, and 2-(4-carboxypiperidino)-4-(3,4-methylenedioxy-benzyl)amino-6-nitroquinazoline, and 2-phenyl-8-ethoxycycloheptimidazole.

Still other type V phosphodiesterase inhibitors useful in conjunction with the present invention include: IC-351 (ICOS); 4-bromo-5-(pyridylmethylamino)-6-[3-(4-chlorophenyl)propoxy]-3(2H)pyridazinone; 1-[4-[(1,3-benzodioxol-5-ylnethyl)amino]-6-chloro-2-quinazolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]-purin-4(3H)one; furazlocillin; cis-2-hexyl-5-methyl-3,4,5,6a,7,8,9,9a-octahydrocyclopent[4,5]imidazo[2,1-b]purin-4-one; 3-acetyl-1-(2-chlorobenzyl)-2-propylindole -6-carboxylate; 4-bromo-5-(3-pyridylmethylamino)-6-(3-(4-chlorophenyl)propoxy)-3-(2H)pyridazinone; 1-methyl-5-(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); and Sch-51866.

Other phosphodiesterase inhibitors that may be used in the method of this invention include nonspecific phosphodiesterase inhibitors such as theophylline, IBMX, pentoxifylline and papaverine, and direct vasodilators such as hydralazine.

A testosterone containing gel, such as AndroGel® is administered to increase and enhance the therapeutic effectiveness of drugs effective at inhibiting the activity of a phosphodiesterase, in either hypogonadal or eugonadal men having erectile dysfunction. While pharmaceuticals such as VIAGRA® work principally by various physiological mechanisms of erection initiation and maintenance, the testosterone gel used in accordance with the present invention plays a beneficial role physiologically, and stimulates both sexual motivation (that is, libido) and sexual performance. Testosterone controls the expression of the nitric oxide synthase gene. See Reilly et al., Androgenic Regulation of NO Availability in Rat Penile Erection, 18 J. Andrology 110 (1997); Park et al., Effects of Androgens on the Expression of Nitric Oxide Synthase mRNAs in Rat Corpous Cavernosum, 83 BJU Int'l. 327 (1999). Thus, testosterone and other androgens clearly play a role in erectile dysfunction. See Lugg et al., The Role of Nitric Oxide in Erectile Function, 16 J. Andrology 2 (1995); Penson et al., Androgen and Pituitary Control of penile Nitric Oxide Synthase and Erectile Function In the Rat, 55 Biology of Reproduction 576 (1996); Traish et al., Effects of Castration and Androgen Replacement on Erectile Function in a Rabbit Model, 140 Endocrinology 1861 (1999). Moreover, testosterone replacement restores nitric oxide activity. See Baba et al. Delayed Testosterone Replacement Restores Nitric Oxide Synthase Containing Nerve Fibres and the Erectile Response in Rat Penis, BJU Int'l 9531 (2000); Garban et al., Restoration of Normal Adult Penile Erectile Response in Aged Rats by Long-Term Treatment with Androgens, 53 Biology of Reproduction 1365 (1995); Marin et al., Androgen-dependent Nitric Oxide Release in Rat Penis Correlates with Levels of Constitutive Nitric Oxide Synthase Isoenzymes, 61 Biology of Reproduction 1012 (1999).

As disclosed herein, adequate blood levels of testosterone are important to erection. The pharmaceutical(s) for erectile dysfunction is taken in accordance with the prescription requirements. For example, VIAGRA® is generally taken 20-40 minutes before sexual intercourse in 50 mg doses. This combination of therapy is particularly useful in hypogonadal men who need increased testosterone levels in order to optimize the effects of VIAGRA® and the sexual experience as a whole. In essence, a synergistic effect is obtained. AndroGel® is preferably applied to the body for a sufficient number of days so that the steady-state levels of testosterone are achieved.

The active agents of the present invention may be administered, if desired, in the form of salts, esters, amides, enantiomers, isomers, tautomers, prodrugs, derivatives and the like, provided the salt, ester, amide, enantiomer, isomer, tautomer, prodrug, or derivative is suitable pharmacologically, that is, effective in the present methods, combinations and compositions. Salts, esters, amides, enantiomers, isomers, tautomers, prodrugs and other derivatives of the active agents may be prepared using standard procedures known to those skilled in the art of synthetic organic chemistry and described, for example, by J. March, Advanced Organic Chemistry; Reactions, Mechanisms and Structure, 4th Ed. (New York: Wiley-Interscience, 1992). For example, acid addition salts are prepared from the free base using conventional methodology, and involves reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, for example, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, for example, hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are halide salts, such as may be prepared using hydrochloric or hydrobromic acids. Conversely, preparation of basic salts of acid moieties which may be present on a phosphodiesterase inhibitor molecule are prepared in a similar manner using a pharmaceutically acceptable base such as sodium hydroxide, potassium hydroxide, ammonium hydroxide, calcium hydroxide, trimethylamine, or the like. Particularly preferred basic salts herein are alkali metal salts, for example, the sodium salt, and copper salts. Preparation of esters involves functionalization of hydroxyl and/or carboxyl groups which may be present within the molecular structure of the drug. The esters are typically acyl-substituted derivatives of free alcohol groups, that is, moieties that are derived from carboxylic acids of the formula RCOOH where R is alkyl, and preferably is lower alkyl. Esters can be reconverted to the free acids, if desired, by using conventional hydrogenolysis or hydrolysis procedures. Amides and prodrugs may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety, which results in a compound that is therapeutically inactive until modified by an individual's metabolic system.

The therapeutic agents of the present invention can be formulated as a single pharmaceutical composition or as independent multiple pharmaceutical compositions. Pharmaceutical compositions according to the present invention include those suitable for oral, percutaneous, transmucosal, implantation, inhalation spray, rectal, vaginal, topical, buccal (for example, sublingual), or parenteral (for example, subcutaneous, intramuscular, intravenous, intramedullary and intradermal injections, or infusion techniques) administration, although the most suitable route in any given case will depend on the nature and severity of the condition being treated and on the nature of the particular compound which is being used. Illustratively, methyltestosterone is administration orally, and testosterone and estradiol are administered percutaneously.

For oral administration, the pharmaceutical composition of methyltestosterone may be in the form of, for example, a tablet, capsule, cachet, lozenge, dispensable powder, granule, solution, suspension, emulsion or liquid. Capsules, tablets, etc., can be prepared by conventional methods well known in the art. Oral formulations can contain excipients such as binders (for example, hydroxypropylmethylcellulose, polyvinyl pyrilodone, other cellulosic materials and starch), diluents (for example, lactose and other sugars, starch, dicalcium phosphate and cellulosic materials), disintegrating agents (for example, starch polymers and cellulosic materials) and lubricating agents (for example, stearates and talc). Solutions, suspensions and powders for reconstitutable delivery systems include vehicles such as suspending agents (for example, gums, zanthans, cellulosics and sugars), humectants (for example, sorbitol), solubilizers (for example, ethanol, water, PEG and propylene glycol), surfactants (for example, sodium lauryl sulfate, Spans, Tweens, and cetyl pyridine), preservatives and antioxidants (for example, parabens, vitamins E and C, and ascorbic acid), anti-caking agents, coating agents, and chelating agents (for example, EDTA).

Percutaneous administration includes transdermal delivery systems that include patches, gels, tapes and creams, and can contain excipients such as alcohols, penetration enhancers, and thickeners, as well as solubilizers (for example propylene glycol, bile salts, and amino acids), hydrophilic polymers (for example, polycarbophil and polyvinylpyrolidone), and adhesives and tackifiers (for example, polyisobutylenes, silicone-based adhesives, acrylates and polybutene).

Transmucosal formulations or delivery systems include patches, tablets, suppositories, pessaries, gels and creams, and can contain excipients such as solubilizers and permeation enhancers (for example, propylene glycol, bile salts and amino acids), and other vehicles (for example, polyethylene glycol, fatty acid esters and derivatives, and hydrophilic polymers such as hydroxypropylmethylcellulose and hyaluronic acid).

Injectable drug formulations include solutions, suspensions, gels, microspheres and polymeric injectables, and can comprise excipients such as solubility-altering agents (for example, ethanol, propylene glycol and sucrose) and polymers (for example, polycaprylactones and PLGA's).

Implantable formulations or systems include rods and discs, and can contain excipients such as PLGA and polycaprylactone.

The therapeutic agents of the present invention can then be administered orally, percutaneously, transmucosally, by implantation, by inhalation spray, rectally, vaginally, topically, buccally or parenterally in dosage unit formulations containing conventional nontoxic pharmaceutically acceptable carriers, adjuvants, and vehicles as desired. The compounds of the present invention can be administered by any conventional means available for use in conjunction with pharmaceuticals, either as individual therapeutic compounds or as a combination of therapeutic compounds.

The compositions of the present invention can be administered for the prophylaxis or treatment of a menopause disorder by any means that produce contact of these compounds with their site of action in the body, for example in the ileum, the plasma, or the liver of a mammal.

Additionally, the methods, combinations and compositions of the present invention may optionally include salts, emollients, stabilizers, antimicrobials, fragrances, and propellants.

In another embodiment, the therapeutic agents come in the form of kits or packages containing a sex hormone binding globulin synthesis inhibiting agent, and one or more steroids, including for example, androgen and/or estrogen. Illustratively, the kits or packages contain methyltestosterone and testosterone, or methyltestosterone and estradiol, or methyltestosterone and testosterone and estradiol, or methyltestosterone and testosterone and a pharmaceutical suitable for treating erectile dysfunction, or methyltestosterone and estradiol and a pharmaceutical suitable for treating erectile dysfunction, or methyltestosterone and testosterone and estradiol and a pharmaceutical suitable for treating erectile dysfunction, in amounts sufficient for the proper dosing of the drugs. In another embodiment, the kits contain methyltestosterone in a dosage form suitable for oral administration, for example, a tablet, and testosterone and/or estradiol in a dosage form suitable for percutaneous administration, for example, a gel or a patch. The therapeutic agents of the present invention can be packaged in the form of kits or packages in which the daily (or other periodic) dosages are arranged for proper sequential or simultaneous administration. The present invention further provides a kit or package containing a plurality of dosage units, adapted for successive daily administration, each dosage unit comprising at least one of the therapeutic agents of the present invention. This drug delivery system can be used to facilitate administering any of the various embodiments of the therapeutic compositions. In one embodiment, the system contains a plurality of dosages to be taken daily via oral administration (as commonly practiced in the oral contraceptive art). In another embodiment, the system contains a plurality of dosages to be administered weekly via transdermal administration (as commonly practiced in the hormone replacement art). In yet another embodiment, the system contains a plurality of dosages to be administered daily, or weekly, or monthly, for example, with one or more therapeutic agents administered orally, and one or more therapeutic agents administered transdermally.

The present invention is further illustrated by the following examples, which should not be construed as limiting in any way. For the below examples, it is understood that ANDROGEL® administered at 5.0 g/day, delivers 50 mg/day of testosterone to the skin of which about 10%, or 5 mg, is absorbed; and ANDROGEL® administered at 10.0 g/day, delivers 100 mg/day of testosterone to the skin of which about 10%, or 5 mg, is absorbed; and estradiol gel (Table 7) administered at 5.0 g/day, delivers 3 mg/day of estrogen to the skin of which about 10%, or 0.3 mg, is absorbed; and estradiol gel administered at 10.0 g/day, delivers 6 mg/day of estrogen to the skin of which about 10%, or 0.6 mg, is absorbed, for 30 days.

### EXAMPLES

### Example 1

In one embodiment of the present invention, the therapeutic combination or kit is comprised of an orally deliverable methyltestosterone and a non-orally deliverable testosterone. In this example, methyltestosterone is formulated as a capsule for oral administration and each dosage unit contains 5 mg of methyltestosterone. Testosterone is formulated as a gel for transdermal administration as described above in Table 6 (ANDROGEL®).

In a prophetic example, 10 males and 10 females age 18 and older experiencing a menopausal disorder will be randomized to receive a daily dose of 5 mg or 20 mg methyltestosterone for 30 days, plus (a) 5.0 g/day ANDROGEL®, or (b) 10 g/day ANDROGEL®. The testosterone gel is rubbed daily onto a given area of skin. Menopausal disorders will be studied prior to treatment, during treatment and following treatment, by methods known in the art. Applicant expects the menopausal disorders will show improvement with the combination.

### Example 2

In one embodiment of the present invention, the therapeutic combination or kit is comprised of an orally deliverable methyltestosterone and a non-orally deliverable estrogen. In this example, methyltestosterone is formulated as a capsule for oral administration and each dosage unit contains 5 mg of methyltestosterone. Estrogen is formulated as a gel for transdermal administration as described above in Table 7.

In a prophetic example, 10 males and 10 females age 18 and older experiencing a menopausal disorder will be randomized to receive a daily dose of 5 mg or 30 mg methyltestosterone for 30 days, plus (a) 5.0 g/day estradiol gel or (b) 10 g/day estradiol gel. The estrogen gel is rubbed daily onto a given area of skin. Menopausal disorders will be studied prior to treatment, during treatment and following treatment, by methods known in the art. Applicant expects the menopausal disorders will show improvement with the combinations.

### Example 3

In one embodiment of the present invention, the therapeutic combination or kit is comprised of an orally deliverable methyltestosterone, a non-orally deliverable testosterone, and a non-orally deliverable estrogen. In this example, methyltestosterone is formulated as a capsule for oral administration and each dosage unit contains 10 mg of methyltestosterone. Testosterone is formulated as a gel for transdermal administration as described above in Table 6 (ANDROGEL®). Estrogen is formulated as a gel for transdermal administration as described above in Table 7.

In a prophetic example, 10 males and 10 females age 18 and older experiencing a menopausal disorder will be randomized to receive a daily dose of 10 mg or 50 mg methyltestosterone for 30 days, plus (a) 5.0 g/day ANDROGEL® and 5.0 g/day estradiol gel or (b) 5.0 g/day ANDROGEL® and 10.0 g/day estradiol gel, or (c) 10.0 g/day ANDROGEL® and 5 g/day estradiol gel, or (d) 10.0 g/day ANDROGEL® and 10.0 g/day estradiol gel. The gels are rubbed daily onto a given area of skin. Menopausal disorders will be studied prior to treatment, during treatment and following treatment by methods known in the art. Applicant expects the menopausal disorders will show improvement with the combination.

### Example 4

In one embodiment of the present invention, the therapeutic combination or kit is comprised of an orally deliverable methyltestosterone, a non-orally deliverable testosterone, and a pharmaceutical agent for treating erectile dysfunction. In this example, methyltestosterone is formulated as a capsule for oral administration and each dosage unit contains 10 mg of methyltestosterone. Testosterone is formulated as a gel for transdermal administration as described above in Table 6 (ANDROGEL®). The pharmaceutical agent for treating erectile dysfunction is VIAGRA® (citrate salt of sildenafil), an inhibitor of cyclic guanosine monophophate-specific phosphodiesterase type 5, and is formulated as a 50 mg tablet for oral administration.

In a prophetic example, 10 males age 18 and older will be randomized to receive a daily dose of 10 mg or 50 mg methyltestosterone for 30 days, plus (a) 5.0 g/day of ANDROGEL® for 30 days plus 50 mg of sildenafil citrate 1 hour before intercourse after at least 1 day of methyltestosterone and ANDROGEL® therapy; or (b) 10.0 g/day of ANDROGEL® for 30 days plus 50 mg of sildenafil citrate 1 hour before intercourse after at least 1 day of methyltestosterone and ANDORGEL® therapy; or (c) 5.0 g/day of ANDROGEL® for 30 days and nothing before intercourse. The gel is rubbed daily onto a given area of skin. Libido, erections and sexual performance will be studied by methods known in the art. Applicant expects that all test parameters will show improvement with the combination.

### Example 5

In one embodiment of the present invention, the therapeutic combination or kit is comprised of an orally deliverable methyltestosterone, a non-orally deliverable estrogen, and a pharmaceutical agent for treating erectile dysfunction. In this example, methyltestosterone is formulated as a capsule for oral administration and each dosage unit contains 10 mg of methyltestosterone. Estrogen is formulated as a gel for transdermal administration as described above in Table 7. The pharmaceutical agent for treating erectile dysfunction is VIAGRA® (citrate salt of sildenafil), an inhibitor of cyclic guanosine monophophate-specific phosphodiesterase type 5, and is formulated as a 50 mg tablet for oral administration.

In a prophetic example, 10 males age 18 and older will be randomized to receive a daily dose of 10 mg or 50 mg methyltestosterone for 30 days, plus (a) 5.0 g/day of estradiol gel for 30 days plus 50 mg of sildenafil citrate 1 hour before intercourse after at least 1 day of methyltestosterone and estradiol gel therapy; or (b) 10.0 g/day of estradiol gel for 30 days plus 50 mg of sildenafil citrate 1 hour before intercourse after at least 1 day of methyltestosterone and estradiol gel therapy; or (c) 5.0 g/day of estradiol gel for 30 days and nothing before intercourse. The gel is rubbed daily onto a given area of skin. Libido, erections and sexual performance will be studied by methods known in the art. Applicant expects that all test parameters will show improvement with the combination.

### Example 6

In one embodiment of the present invention, the therapeutic combination or kit is comprised of an orally deliverable methyltestosterone, a non-orally deliverable testosterone, a non-orally deliverable estrogen and a pharmaceutical agent for treating erectile dysfunction. In this example, methyltestosterone is formulated as a capsule for oral administration and each dosage unit contains 10 mg of methyltestosterone. Testosterone is formulated as a gel for transdermal administration as described above in Table 6 (ANDROGEL®). Estrogen is formulated as a gel for transdermal administration as described above in Table 7. The pharmaceutical agent for treating erectile dysfunction is VIAGRA® (citrate salt of sildenafil), an inhibitor of cyclic guanosine monophophate-specific phosphodiesterase type 5, and is formulated as a 50 mg tablet for oral administration.

In a prophetic example, 10 males age 18 and older will be randomized to receive a daily dose of 10 mg or 50 mg of methyltestosterone for 30 days, plus (a) 5.0 g/day ANDROGEL® and 5.0 g/day estradiol gel for 30 days plus 50 mg of sildenafil citrate 1 hour before intercourse after at least 1 day of methyltestosterone, ANDROGEL® and estradiol gel therapy; or (b) 5.0 g/day ANDROGEL® and 10.0 g/day estradiol gel for 30 days plus 50 mg of sildenafil citrate 1 hour before intercourse after at least 1 day of methyltestosterone and ANDORGEL® therapy; or (c) 10.0 g/day ANDROGEL® and 5.0 g/day estradiol gel for 30 days plus 50 mg of sildenafil citrate 1 hour before intercourse after at least 1 day of methyltestosterone, ANDROGEL® and estradiol gel therapy; (d) 10.0 g/day ANDROGEL® and 10.0 g/day estradiol gel for 30 days plus 50 mg of sildenafil citrate 1 hour before intercourse after at least 1 day of methyltestosterone, ANDROGEL® and estradiol gel therapy; or (e) 5.0 g/day ANDROGEL® and 5.0 g/day estradiol gel for 30 days and nothing before intercourse; or (f) 5.0 g/day ANDROGEL® and 10.0 g/day estradiol gel for 30 days and nothing before intercourse; or (g) 10.0 g/day ANDROGEL® and 5.0 g/day estradiol gel for 30 days and nothing before intercourse; or (h) 10.0 g/day ANDROGEL® and 10.0 g/day estradiol gel for 30 days and nothing before intercourse. The gels are rubbed daily onto a given area of skin. Libido, erections and sexual performance will be studied by methods known in the art. Applicant expects that all test parameters will show improvement with the combination.

The practice of the present invention will employ, unless otherwise indicated, conventional techniques of pharmacology and pharmaceutics, which are within the skill of the art.

All cited literature and patent references are hereby incorporated herein by reference. Although the invention has been described with respect to specific embodiments and examples, it should be appreciated that other embodiments utilizing the concept of the present invention are possible without departing from the scope of the invention. The claimed elements, and any and all modifications, variations or equivalents that fall within the true spirit and scope of the underlying principles define the present invention.

What is claimed is

1. A method of treating, preventing or reducing the risk of developing a menopause disorder in a mammal in need thereof, comprising administering to the mammal a menopause disorder effective amount of an orally deliverable pharmaceutically-acceptable sex hormone binding globulin synthesis inhibiting agent, and at least one of a non-orally deliverable pharmaceutically-acceptable steroid.

2. The method of claim 1 wherein the sex hormone binding globulin synthesis inhibiting agent is methyltestosterone.

3. The method of claim 2 wherein the methyltestosterone is administered in the form of a tablet, capsule, cachet, lozenge, dispensable powder, granule, solution, suspension, emulsion or liquid.

4. The method of claim 1 wherein the non-orally deliverable steroid is at least one of an androgen or an estrogenic steroid.

5. The method of claim 4 wherein the androgen is a steroid in the testosterone synthetic pathway.

6. The method of claim 5 wherein the steroid is at least one of testosterone, androstenedione, androstenediol, dehydroepiandrosterone, prenenolone, and dihydrotestosterone.

7. The method of claim 6 wherein the steroid is testosterone.

8. The method of claim 7 wherein the androgen is administered percutaneously.

9. The method of claim 8 wherein the androgen is administered in the form of a hydroalcoholic gel.

10. The method of claim 9 wherein the hydroalcoholic gel further comprises at least one of a lower alcohol, a penetration enhancer, and a thickener.

11. The method of claim 10 wherein the lower alcohol is selected from the group consisting of ethanol, 2-propanol, and mixtures thereof.

12. The method of claim 10 wherein the enhancer is isopropyl myristate.

13. The method of claim 10 wherein the thickener is CARBOPOL®.

14. The method of claim 4 wherein the estrogenic steroid is estradiol.

15. The method of claim 14 wherein the estrogenic steroid is administered percutaneously.

16. The method of claim 15 wherein the estrogenic steroid is administered in the form of a hydroalcoholic gel.

17. The method of claim 16 wherein the hydroalcoholic gel further comprises at least one of a lower alcohol, and a thickener.

18. The method of claim 17 wherein the lower alcohol is at least one of ethanol, 2-propanol, and mixtures thereof.

19. The method of claim 18 wherein the thickener is CARBOPOL®.

20. The method of claim 1 wherein the sex hormone binding globulin synthesis inhibiting agent and the steroid are each provided as a separate component of a kit.

21. The method of claim 1 wherein the mammal is a human.

22. The method of claim 1 wherein the sex hormone binding globulin synthesis inhibiting agent and the steroid are administered in a sequential manner.

23. The method of claim 1 wherein the sex hormone binding globulin synthesis inhibiting agent and the steroid are administered in a substantially simultaneous manner.

24. The method of claim 1 further comprising at least one of a pharmaceutical agent for treating erectile dysfunction.

25. The method of claim 24 wherein the pharmaceutical agent is at least one of sildenafil citrate, pentoxifylline, yohimbine hydrocholoride, apomorphine, alprostadil, papavaerine, and phentolamine.

26. The method of claim 1 where the sex hormone binding globulin synthesis inhibiting agent comprises about 0.2 mg to about 50.0 mg methyltestosterone, the steroid comprises about 0.1 g to about 100.0 g testosterone.

27. The method of claim 26 wherein the mammal achieve hormonal steady state levels of testosterone.

28. The method of claim 1 where the sex hormone binding globulin synthesis inhibiting agent comprises about 0.2 mg to about 50.0 mg methyltestosterone, the steroid comprises about 0.1 g to about 100.0 g estradiol.

29. The method of claim 28 wherein the mammal achieve hormonal steady state levels of estradiol.

30. A kit comprising an orally deliverable sex hormone binding globulin synthesis inhibiting agent and at least one of a non-orally deliverable steroid, wherein the sex hormone binding globulin synthesis inhibiting agent and the steroid together make a menopause disorder effective amount.

31. The kit of claim 30 wherein the sex hormone binding globulin synthesis inhibiting agent is methyltestosterone.

32. The kit of claim 30 wherein the sex hormone binding globulin synthesis inhibiting agent is administered in the form of a tablet, capsule, cachet, lozenge, dispensable powder, granule, solution, suspension, emulsion or liquid.

33. The kit of claim 30 wherein the steroid is administered percutaneously.

34. The kit of claim 30 wherein the steroid is a steroid in the testosterone synthetic pathway.

35. The kit of claim 30 wherein the steroid is selected from the group consisting of testosterone, androstenedione, androstenediol, dehydroepiandrosterone, prenenolone, and dihydrotestosterone.

36. The kit of claim 35 wherein the steroid is testosterone.

37. The kit of claim 36 wherein the testosterone is administered percutaneously.

38. The kit of claim 30 wherein the steroid is an estrogenic steroid.

39. The kit of claim 38 wherein the estrogenic steroid is estradiol.

40. The kit of claim 39 wherein the testosterone is administered percutaneously.

41. The kit of claim 30 wherein the sex hormone binding globulin synthesis inhibiting agent is present in an amount from about 0.2 mg to about 50.0 mg.

42. The kit of claim 30 wherein the steroid is present in an amount from about 0.1 mg to about 100.0 mg.

43. The kit of claim 30 further comprising at least one of a pharmaceutical agent for treating erectile dysfunction.

44. The kit of claim 43 wherein the agent for treating erectile dysfunction is selected from the group consisting of sildenafil citrate, pentoxifylline, yohimbine hydrocholoride, apomorphine, alprostadil, papavaerine, and phentolamine.

45. A method of treating, preventing or reducing the risk of developing a menopause disorder in a mammal in need thereof, comprising administering to the mammal in a combination therapy an orally deliverable pharmaceutically-acceptable sex hormone binding globulin synthesis inhibiting agent, and at least one of a non-orally deliverable pharmaceutically-acceptable steroids, wherein the amount of the sex hormone binding globulin synthesis inhibiting agent and the steroid together make a menopause disorder effective amount.

46. The method of claim 45 wherein the sex hormone binding globulin synthesis inhibiting agent is methyltestosterone.

47. The method of claim 46 wherein the methyltestosterone is administered in the form of a tablet, capsule, cachet, lozenge, dispensable powder, granule, solution, suspension, emulsion or liquid.

48. The method of claim 47 wherein the non-orally deliverable steroid is at least one of an androgen or an estrogenic steroid.

49. The method of claim 48 wherein the androgen is a steroid in the testosterone synthetic pathway.

50. The method of claim 49 wherein the steroid is at least one of testosterone, androstenedione, androstenediol, dehydroepiandrosterone, prenenolone, and dihydrotestosterone.

51. The method of claim 50 wherein the steroid is testosterone.

52. The method of claim 51 wherein the androgen is administered percutaneously.

53. The method of claim 52 wherein the androgen is administered in the form of a hydroalcoholic gel.

54. The method of claim 53 wherein the hydroalcoholic gel further comprises at least one of a lower alcohol, a penetration enhancer, and a thickener.

55. The method of claim 54 wherein the lower alcohol is selected from the group consisting of ethanol, 2-propanol, and mixtures thereof.

56. The method of claim 54 wherein the enhancer is isopropyl myristate.

57. The method of claim 54 wherein the thickener is CARBOPOL®.

58. The method of claim 48 wherein the estrogenic steroid is estradiol.

59. The method of claim 58 wherein the estrogenic steroid is administered percutaneously.

60. The method of claim 59 wherein the estrogenic steroid is administered in the form of a hydroalcoholic gel.

61. The method of claim 60 wherein the hydroalcoholic gel further comprises at least one of a lower alcohol, and a thickener.

62. The method of claim 61 wherein the lower alcohol is at least one of ethanol, 2-propanol, and mixtures thereof.

63. The method of claim 62 wherein the thickener is CARBOPOL®.

64. The method of claim 45 wherein the sex hormone binding globulin synthesis inhibiting agent and the steroid are each provided as a separate component of a kit.

65. The method of claim 45 wherein the mammal is a human.

66. The method of claim 45 wherein the sex hormone binding globulin synthesis inhibiting agent and the steroid are administered in a sequential manner.

67. The method of claim 45 wherein the sex hormone binding globulin synthesis inhibiting agent and the steroid are administered in a substantially simultaneous manner.

68. The method of claim 45 further comprising at least one of a pharmaceutical agent for treating erectile dysfunction.

69. The method of claim 64 wherein the pharmaceutical agent is at least one of sildenafil citrate, pentoxifylline, yohimbine hydrocholoride, apomorphine, alprostadil, papavaerine, and phentolamine.

70. The method of claim 41 where the sex hormone binding globulin synthesis inhibiting agent comprises about 0.2 mg to about 50.0 mg methyltestosterone, the steroid comprises about 0.1 g to about 100.0 g testosterone.

71. The method of claim 70 wherein the mammal achieve hormonal steady state levels of testosterone.

72. The method of claim 41 where the sex hormone binding globulin synthesis inhibiting agent comprises about 0.2 mg to about 50.0 mg methyltestosterone, the steroid comprises about 0.1 g to about 100.0 g estradiol.

73. The method of claim 72 wherein the mammal achieve hormonal steady state levels of estradiol.
